(19) United States
(12) Patent Application Publication
(10) Pub. No.: US 2026/0285959 A1
(43) Pub. Date: Sep. 24, 2026

(54) ANTI-PCNA NANOBODIES, FUSION PROTEINS, AND USES THEREOF

(71) Applicant: Hubei University, Wuhan (CN)

(72) Inventors: Lixin MA, Wuhan (CN); Fei WANG, Wuhan (CN); Shuai ZHAO, Wuhan (CN); Yang WANG, Wuhan (CN); Fang YU, Wuhan (CN); Jingwei ZENG, Wuhan (CN); Wanping CHEN, Wuhan (CN)

(73) Assignee: Hubei University, Wuhan (CN)

(21) Appl. No.: 19/571,577

(22) Filed: Mar. 19, 2026

(30) Foreign Application Priority Data
Mar. 19, 2025 (CN) 202510328163.0

(51) Int. Cl. C07K 16/18

(52) U.S. Cl. CPC C07K 16/18; C07K 2317/565; C07K 2317/567; C07K 2317/569; C07K 2317/82; C07K 2319/02; C07K 2319/21; C07K 2319/70

(57) ABSTRACT

The present disclosure provides anti-PCNA nanobodies, fusion proteins comprising the same, and uses thereof, which belong to the field of biotechnology. In the present disclosure, artificial intelligence technology is employed to design nanobodies that specifically target PCNA. The nanobodies exhibit favorable specificity and high affinity, and are capable of efficiently binding to the PCNA antigen. Further, rational design of the nanobodies via artificial intelligence technology yields nanobody mutants with higher affinity. Moreover, fusion proteins are prepared by fusing the aforementioned nanobodies or nanobody mutants with an RBCC domain or its mutant. The fusion proteins can specifically bind to a target protein via their nanobody domains, thereby initiating a protein degradation pathway to achieve targeted degradation of PCNA protein, and they can also activate intracellular p53, inducing its phosphorylation to prevent ubiquitination-mediated degradation by ubiquitin ligases such as MDM2, demonstrating significant clinical application value.

### CROSS-REFERENCE TO RELATED APPLICATIONS

The application claims priority to Chinese patent application No. 202510328163.0, filed on Mar. 19, 2025, with the entire contents incorporated herein by reference.

### SEQUENCE LISTING

The sequence listing xml file submitted herewith, named “Sequence_Listing.xml”, created on Mar. 19, 2026, and having a file size of 45,927 bytes, is incorporated by reference herein.

### TECHNICAL FIELD

The present disclosure relates to the technical field of biotechnology, and in particular to anti-PCNA nanobodies, fusion proteins comprising the same, and uses thereof.

### BACKGROUND

The Proliferating Cell Nuclear Antigen (PCNA) gene encodes a nuclear protein that is primarily involved in DNA replication and repair processes. PCNA forms a trimeric ring structure that slides along DNA strands and acts as a loading factor for DNA polymerases, ensuring the high efficiency and accuracy of DNA replication. PCNA plays a pivotal role in cell proliferation, and its expression level is closely correlated with the cellular proliferation rate. During the initiation and elongation phases of DNA replication, PCNA functions as a sliding clamp which interacts with various replication and repair proteins to ensure the high efficiency and accuracy of DNA replication. PCNA is also involved in multiple DNA repair pathways, including base excision repair (BER), nucleotide excision repair (NER), and mismatch repair (MMR). These repair mechanisms are essential for maintaining genomic stability and preventing the accumulation of mutations.

PCNA is highly expressed in various cancers, and its expression level is positively correlated with the proliferation rate and malignancy of tumor cells. Owing to its pivotal role in DNA replication and repair, aberrant expression or dysfunction of PCNA can induce genomic instability, thereby promoting the development and progression of cancer. Therefore, PCNA has become an important therapeutic and research target for cancer.

Existing PCNA inhibitors primarily inhibit DNA replication and repair by impeding the interaction of PCNA with DNA polymerases or other repair proteins. However, research on PCNA degraders remains relatively limited. Currently available PCNA inhibitors exhibit limited efficacy and may be accompanied by a high risk of side effects. Thus, there is a critical need to develop more efficient and specific PCNA-targeted degradation technologies.

Nanobodies are single-domain antibodies derived from the heavy-chain antibodies of camelids, which retain the specificity and affinity of conventional antibodies while possessing a smaller molecular mass and higher stability. Nanobodies are capable of penetrating tissues and cells and binding to target proteins more readily, thus having broad application potential in biomedical research and therapy.

Targeted Protein Degradation (TPD) technology utilizes the intracellular ubiquitin-proteasome system and lysosomal degradation system to achieve specific and efficient degradation of disease-associated proteins, thereby attaining therapeutic effects for diseases. Compared with traditional small-molecule inhibitors, TPD offers multiple advantages: it is event-driven, requires a low therapeutic dose and exerts its effects at only a catalytic dose; moreover, it can target some undruggable proteins.

TRIMbody is an emerging TPD technology generated by fusing a nanobody to TRIM21, a member of the Tripartite motif-containing (TRIM) protein family. TRIM21 is an E3 ubiquitin ligase that can specifically recognize and bind to target proteins, thereby promoting their ubiquitination and ultimately mediate their degradation in the proteasome. By combining the high specificity of nanobodies with the efficient degradation activity of TRIM21, TRIMbody technology provides a more efficient and precise approach for PCNA degradation, which is hold the potential to enhance the therapeutic effect for PCNA-related cancers.

However, to date, no research has been reported on nanobodies targeting PCNA.

### SUMMARY

The present disclosure provides anti-PCNA nanobodies, fusion proteins comprising the same, and uses thereof. In the present disclosure, artificial intelligence technology is employed to design nanobodies that specifically target PCNA. The resulting nanobodies exhibit favorable specificity and high affinity. Further, the nanobodies is fused with an RBCC domain or its mutant to obtain fusion proteins. Such fusion proteins can efficiently and precisely degrade PCNA. Therefore, the nanobodies and fusion proteins hold promising application prospects in the prevention and/or treatment of diseases associated with the PCNA target.

In a first aspect, the present disclosure provides a nanobody against proliferating cell nuclear antigen (PCNA) protein, which comprises three complementarity determining regions (CDRs): CDR1, CDR2 and CDR3; wherein CDR1 has an amino acid sequence as shown in SEQ ID NO: 1, CDR2 has an amino acid sequence as shown in SEQ ID NO: 2, and CDR3 has an amino acid sequence as shown in any one of SEQ ID NOs: 3-6.

In the present disclosure, the term “nanobody”, also known as a single-domain antibody (sdAb) or a VHH antibody, refers to an antibody composed of a heavy chain variable region (VHH). The antigen-binding activity and stability of a nanobody are substantially identical to those of a conventional antibody. A nanobody is composed sequentially of framework region 1 (FR1), complementarity determining region 1 (CDR1), framework region 2 (FR2), complementarity determining region 2 (CDR2), framework region 3 (FR3), complementarity determining region 3 (CDR3), and framework region 4 (FR4). A nanobody may be truncated at the N-terminus or C-terminus such that it contains only a portion of FR1 and/or FR4, or may lack one or two of the framework regions, provided that it substantially retains antigen-binding activity and specificity. Among these regions, the complementarity determining regions are primarily responsible for antigen binding.

In some embodiments, the nanobody further comprises four framework regions (FRs): FR1, FR2, FR3 and FR4; wherein FR1 has an amino acid sequence as shown in SEQ ID NO: 7, FR2 has an amino acid sequence as shown in SEQ ID NO: 8, FR3 has an amino acid sequence as shown in SEQ ID NO: 9, and FR4 has an amino acid sequence as shown in SEQ ID NO: 10.

It will be appreciated that framework regions are highly conserved. Accordingly, conventional framework regions known in the art may be selected according to practical needs, provided that they enhance the stability of the nanobody.

In some embodiments, the nanobody is selected from any one of the following:
A1) an amino acid sequence as shown in any one of SEQ ID NOs: 11-14;
A2) an amino acid sequence having one or several amino acid substitutions, deletions, or additions compared to the amino acid sequence defined in A1);
A3) an amino acid sequence having at least 80% sequence identity to the amino acid sequence defined in A1) or A2);
A4) an amino acid sequence obtained by linking a tag or a signal peptide to the N-terminus and/or C-terminus of the amino acid sequence defined in A1), A2) or A3).

In the present disclosure, nanobodies that specifically target PCNA were designed via artificial intelligence technology. Said nanobodies exhibit favorable specificity and high affinity, and are capable of efficiently binding to the PCNA antigen.

In a second aspect, the present disclosure provides a mutant of the nanobody against proliferating cell nuclear antigen (PCNA) protein, wherein the nanobody mutant is selected from any one of the following:
B1) an amino acid sequence as shown in any one of SEQ ID NOs: 15-17;
B2) an amino acid sequence having one or several amino acid substitutions, deletions, or additions compared to the amino acid sequence defined in B1);
B3) an amino acid sequence having at least 80% sequence identity to the amino acid sequence defined in B1) or B2);
B4) an amino acid sequence obtained by linking a tag or a signal peptide to the N-terminus and/or C-terminus of the amino acid sequence defined in B1), B2), or B3);
wherein, in B1), the amino acid sequence as shown in SEQ ID NO: 15 is obtained by mutating the amino acid sequence as shown in SEQ ID NO: 11 at position 102 from L to W (L102W); the amino acid sequence as shown in SEQ ID NO: 16 is obtained by mutating the amino acid sequence as shown in SEQ ID NO: 11 at position 101 from S to M (S101M); the amino acid sequence as shown in SEQ ID NO: 17 is obtained by mutating the amino acid sequence as shown in SEQ ID NO: 14 at least at position 113 from H to K (H113K).

In the present disclosure, nanobody mutants with higher affinity were obtained by rational design of the nanobodies using artificial intelligence technology.

In some embodiments, in addition to the mutation occurring at one of positions 101, 102, or 113 as described above, the nanobody mutants may further have conservative amino acid substitutions at other positions, such that the mutated nanobodies (e.g., those having the amino acid sequence of SEQ ID NO: 11 or 14) exhibit higher affinity. Preferably, the conservative amino acid substitutions maintain the high affinity of the nanobodies of the present disclosure. It will be apparent to those skilled in the art that such substitutions can occur in regions other than the positions mentioned above while still retaining the corresponding affinity. Preferably, the conservative substitution variants comprise at least one conservative amino acid substitution. Examples of conservative substitutions include substitutions within the following groups of amino acids: basic amino acids (e.g., arginine, lysine, histidine), acidic amino acids (e.g., glutamic acid, aspartic acid), polar amino acids (e.g., glutamine, asparagine), hydrophobic amino acids (e.g., leucine, isoleucine, valine), aromatic amino acids (e.g., phenylalanine, tryptophan, tyrosine), and small amino acids (e.g., glycine, alanine, serine, threonine, methionine).

In a third aspect, the present disclosure provides a fusion protein, which is prepared by fusing any one of the aforementioned nanobodies or nanobody mutants to an RBCC domain or its mutant via a second linker; wherein the amino acid sequence of the second linker is shown in SEQ ID NO: 29, the amino acid sequence of the RBCC domain is shown in SEQ ID NO: 27, and the amino acid sequence of the RBCC domain mutant is shown in SEQ ID NO: 28.

In the present disclosure, the aforementioned nanobodies or nanobody mutants with high affinity and favorable specificity are fused with an RBCC domain or a mutant thereof (a truncated TRIM family protein) to obtain fusion proteins. The resulting fusion proteins can specifically bind to a target protein via their nanobody domains, thereby initiating a protein degradation pathway to achieve targeted degradation of PCNA protein. Meanwhile, said fusion proteins can also activate intracellular p53, and induce phosphorylation thereof to prevent p53 from being ubiquitinated and degraded by ubiquitin ligases such as MDM2, thereby promoting apoptosis and inhibiting cancer cell proliferation. The nanobodies, nanobody mutants, and fusion proteins of the present disclosure therefore exhibit significant clinical application value.

It will be appreciated that linker peptides may be selected from conventional linkers known in the art according to practical needs, provided that they allow for sufficient fusion of the two proteins.

In some embodiments, the fusion protein is selected from any one of the following: C1) an amino acid sequence as shown in any one of SEQ ID NOs: 33-41;
C2) an amino acid sequence having one or several amino acid substitutions, deletions, or additions compared to the amino acid sequence defined in C1);
C3) an amino acid sequence having at least 80% sequence identity to the amino acid sequence defined in C1) or C2);
C4) an amino acid sequence obtained by linking a tag or a signal peptide to the N-terminus and/or C-terminus of the amino acid sequence defined in C1), C2), or C3).

The aforementioned nanobodies, nanobody mutants, and fusion proteins provided may be active polypeptides that are natural, recombinant, or synthetic. Such active polypeptides may be naturally purified products, chemically synthesized products, or products produced from a prokaryotic host (e.g., Escherichia coli) or a eukaryotic host (e.g., yeast, higher plant) using recombinant technology.

In the present disclosure, in A4), B4) or C4), the linking may be performed either directly via a peptide bond or via a linker, using methods conventional in the art. Tags include, but are not limited to: GST (glutathione S-transferase) tag, Trx (thioredoxin) tag, His tag (His-tag), Flag tag, LacZ tag, GFP (green fluorescent protein), sfGFP (superfolder green fluorescent protein), and HA tag (hemagglutinin tag). Those skilled in the art may select an appropriate tag according to practical needs. Use of the tag does not alter the function of the target protein (nanobody, nanobody mutant or fusion protein), as it is intended for isolation, purification, detection, or tracing. The tag may be cleaved from the target protein (nanobody, nanobody mutant or fusion protein) by chemical cleavage or enzymatic methods known in the art (e.g., introducing a protease cleavage site to enable tag removal by cleavage with TEV protease).

In a fourth aspect, the present disclosure provides a biological material, which is selected from any one of the following:
D1) a nucleic acid molecule encoding any one of the aforementioned nanobodies, any one of the aforementioned nanobody mutants, or any one of the aforementioned fusion proteins;
D2) a recombinant vector comprising the nucleic acid molecule of D1);
D3) a recombinant cell comprising the nucleic acid molecule of D1) or the recombinant vector of D2).

In some embodiments, the nucleic acid molecule defined in D1) is selected from any one of the following:
E1) a nucleic acid molecule having a nucleotide sequence as shown in any one of SEQ ID NOs: 18-24;
E2) a nucleic acid molecule that hybridizes to the nucleic acid molecule defined in E1) under stringent conditions and encodes any one of the aforementioned nanobodies, nanobody mutants, or fusion proteins;
E3) a nucleic acid molecule having at least 90% sequence identity to the nucleic acid molecule defined in E1) or E2) and encoding any one of the aforementioned nanobodies, nanobody mutants, or fusion proteins.

The nucleic acid molecules provided herein may be DNA, such as cDNA, genomic DNA, or recombinant DNA; or RNA, such as mRNA or hnRNA. Such nucleic acid molecules may generally be obtained by PCR amplification or artificial synthesis.

The aforementioned recombinant vectors include cloning vectors and expression vectors. Cloning vectors are used for replicating related sequences, and expression vectors are used for expressing related genes. The vector used for constructing an expression vector can be at least one selected from pET23a and pcDNA3.1.

In some embodiments, the method for preparing the aforementioned recombinant cell comprises the step of transforming the aforementioned recombinant vector into an expression host cell.

In the present disclosure, the expression host cell is a conventional host cell in the art, so long as it allows stable self-replication of the recombinant vector and effective expression of the genes carried therein. It may be a prokaryotic cell or a eukaryotic cell, such as Escherichia coli (E. coli) or yeast. Examples of E. coli include E. coli BL21 (DE3), Rosetta (DE3), and BL21 (DE3) plysS. In the present disclosure, E. coli BL21 (DE3) is preferred.

In a fifth aspect, the present disclosure provides use of any one of the aforementioned nanobodies, any one of the aforementioned nanobody mutants, any one of the aforementioned fusion proteins, or the aforementioned biological materials in any one of the following:
F1) use in the preparation of a product for the prophylaxis and/or treatment of a PCNA target-related disease;
F2) use in the preparation of a product for the screening, diagnosis or auxiliary diagnosis of a PCNA target-related disease;
wherein the PCNA target-related disease is a PCNA-positive tumor.

In the present disclosure, “PCNA-positive tumor” refers to a tumor in which PCNA expression is detected in tumor cells. Examples of such tumors include common cancers such as lung cancer, gastric cancer, liver cancer, breast cancer, prostate cancer, colorectal cancer, and esophageal cancer.

In a sixth aspect, the present disclosure provides a pharmaceutical composition for the prophylaxis and/or treatment of a PCNA target-related disease, which comprises any one of the aforementioned nanobodies, any one of the aforementioned nanobody mutants, any one of the aforementioned fusion proteins, or the aforementioned biological material, and a pharmaceutically acceptable carrier.

In the present disclosure, the term “pharmaceutically acceptable carrier” refers to an excipient widely used in the field of pharmaceutical manufacturing. Excipients are primarily used to provide a safe, stable, and functional pharmaceutical composition. They may also enable a desired dissolution rate of the active ingredient after administration to a subject, or facilitate effective absorption of the active ingredient by the subject after administration of the composition. The pharmaceutical excipient may be an inert filler, or may provide a certain function, such as stabilizing the overall pH of the composition or preventing degradation of the active ingredients of the composition.

The pharmaceutical composition of the present disclosure can be prepared by any method known to those skilled in the art based on the present disclosure. Such methods include, but are not limited to, conventional mixing, dissolving, granulating, emulsifying, comminuting, encapsulating, embedding or lyophilizing processes.

In some embodiments, the dosage form of the pharmaceutical composition includes at least one of a solid formulation, a semi-solid formulation, and a liquid formulation.

The pharmaceutical composition provided by the present disclosure may be administered in any form, including injection (intravenous), mucosal, oral (solid and liquid preparations), inhalation, ocular, rectal, topical or parenteral (infusion, injection, implantation, subcutaneous, intravenous, intraarterial, intramuscular) administration. The pharmaceutical composition of the present disclosure may also be in a controlled-release or sustained-release dosage form (e.g., a liposome or microsphere).

In a seventh aspect, the present disclosure provides a method for preparing any one of the aforementioned nanobodies, any one of the aforementioned nanobody mutants, or any one of the aforementioned fusion proteins, comprising the steps of:
culturing the aforementioned recombinant cell to obtain a culture after induced expression; and
isolating the nanobody, nanobody mutant, or fusion protein from the culture.

In the present disclosure, there are no particular limitations on the culture method, culture conditions, and culture medium, so long as the recombinant cell can grow normally. Methods for isolating any one of the aforementioned nanobodies, nanobody mutants, or fusion proteins from the culture are all conventional methods in the art.

The beneficial effects of the present disclosure are as follows:

Different from the prior art, the present disclosure employs artificial intelligence technology to design nanobodies that specifically target PCNA. The nanobodies exhibit favorable specificity and high affinity, and are capable of efficiently binding to the PCNA antigen. Further, rational design of the nanobodies via artificial intelligence technology yields nanobody mutants with higher affinity. Moreover, fusion proteins are prepared by fusing the aforementioned nanobodies or nanobody mutants with an RBCC domain or its mutant. The fusion proteins can specifically bind to a target protein via their nanobody domains, thereby initiating a protein degradation pathway to achieve targeted degradation of PCNA protein; Meanwhile, they can also activate intracellular p53, inducing its phosphorylation to prevent ubiquitination-mediated degradation by ubiquitin ligases such as MDM2, thereby promoting apoptosis and inhibiting cancer cell proliferation. The present disclosure holds significant clinical application value.

### BRIEF DESCRIPTION OF DRAWINGS

FIG. 1 is a schematic diagram showing the element arrangement of the recombinant expression vectors constructed in the present disclosure;

FIG. 2 shows the SDS-PAGE detection results of nanobodies (a) and PCNA antigen (b) in Example 2 of the present disclosure, wherein in panel (a), lane M: Marker, lanes 1-9: PN1-9, respectively; and in panel (b), lane M: Marker, lanes 1-2: PCNA;

FIG. 3 shows the ELISA results of the affinity between nanobodies and PCNA antigen in Example 3 of the present disclosure;

FIG. 4 shows the BL1 results of the affinity between nanobodies PN2 (A), PN6 (B), PN7 (C), PN8 (D) and PCNA antigen in Example 4 of the present disclosure, respectively;

FIG. 5 shows the binding energy and total score results calculated by Rosetta for PN2-WT and mutant PN2M (a and b), and for PN8-WT and mutant PN8M (c and d) with PCNA antigen in Example 5 of the present disclosure, respectively;

FIGS. 6A-6B show the energy change results before and after the PN2L102W mutation calculated by Amber in Example 5 of the present disclosure;

FIG. 6C shows the energy change results before and after the PN8H113K mutation calculated by Amber in Example 5 of the present disclosure;

FIG. 7 shows the ELISA results of the affinity between nanobody (a), nanobody mutants (b) and PCNA antigen in Example 5 of the present disclosure, respectively;

FIG. 8 shows the ELISA results of the affinity between nanobody PN8 (a), its mutant PN8H113K (b) and PCNA antigen in Example 5 of the present disclosure, respectively;

FIG. 9 shows the ELISA results of the affinity between nanobody PN2 (a), its mutants PN2L102W (b), PN2S101M (c), PN2S101F (d) and PCNA antigen in Example 5 of the present disclosure, respectively;

FIG. 10 shows the Western blot results of targeted PCNA protein degradation by fusion proteins (RBCC-PN8 and RBCC-PN8M (a), RS79β-PN8 and RS79E-PN8M (b)) in Example 6 of the present disclosure;

FIG. 11 shows the Western blot results of targeted PCNA protein degradation by fusion proteins (RBCC-PN2M1, RBCC-PN2M2) in Example 6 of the present disclosure;

FIG. 12 shows the Western blot results (a) verifying the pathway of targeted PCNA protein degradation by fusion proteins and phosphorylation level results (b) in Example 6 of the present disclosure.

### DETAILED DESCRIPTION OF THE EMBODIMENTS

The technical solutions in the embodiments of the present disclosure will be clearly and fully described below in conjunction with the accompanying examples. It will be apparent that the embodiments described are merely some, rather than all, embodiments of the present disclosure. Other embodiments obtained by those of ordinary skill in the art without creative efforts based on the embodiments of the present disclosure shall fall within the scope of protection of the present disclosure.

Experimental methods for which specific conditions are not indicated in the following examples are usually performed according to conventional experimental methods in the field of molecular biology, including, but not limited to, those described in Molecular Cloning: A Laboratory Manual by M. R. Green, Molecular Biology by Robert F. Weaver, or as recommended by the manufacturers of kits and instruments. Reagents and biological materials used in the examples are commercially available unless otherwise specified.

### Example 1: Using Artificial Intelligence to Design Nanobodies

First, artificial intelligence technology combined with bioinformatics tools (e.g., SEPPA, MetMHCpan, etc.) was employed to predict hotspot epitopes of the PCNA antigen. These epitopes were then linearly fragmented to generate fragments comprising at least four consecutive residues. Subsequently, CDR-like fragments matching the above fragments were screened from the AbAg database. Structural superimposition and optimization were further performed between the CDR-like fragments and the epitope fragments to construct CDR fragments capable of specifically binding to the PCNA antigen. Then, appropriate framework region (FR) fragments were selected (the amino acid sequences of the CDR fragments and FR fragments are shown in Table 1 below). Finally, a series of nanobodies targeting specific epitopes of the PCNA antigen were obtained and named nanobodies PN1-9, respectively.

TABLE 1
Amino acid sequences of CDR fragments
and FR fragments

| Segment | Amino Acid Sequence |
|---|---|
| CDR1 | GFNIKDT |
| | (SEQ ID NO: 1) |
| CDR2 | YPTSGY |
| | (SEQ ID NO: 2) |
| CDR3 | GSLQKGYLEEFDY |
| | (SEQ ID NO: 3) |
| | GSAVLQKGYFPEEFDY |
| | (SEQ ID NO: 4) |
| | GSQKKITDYFEEFDY |
| | (SEQ ID NO: 5) |
| | GSAVLQKKITDYFHPKKEEFDY |
| | (SEQ ID NO: 6) |
| FR1 | MEVQLEESGGGLVQPGGSLRLSCAAS |
| | (SEQ ID NO: 7) |
| FR2 | YIGWVRQAPGKGEEWVASI |
| | (SEQ ID NO: 8) |
| FR3 | TRYADSVKGRFTISADTSKNTAYLQM |
| | NSLRAEDTAVYYCAA |
| | (SEQ ID NO: 9) |
| FR4 | WGQGTLVTVSS |
| | (SEQ ID NO: 10) |

Exemplarily, the amino acid sequence of nanobody PN2 is shown below:

(SEQ ID NO: 11)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSLQKGYLEEFDYWGQGTLVTVSS.

The underlined portions represent complementarity determining regions CDR1, CDR2, and CDR3, respectively.

Exemplarily, the amino acid sequence of nanobody PN6 is shown below:

(SEQ ID NO: 12)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSAVLQKGYFPEEFDYWGQGTLVTVSS.

The underlined portions represent complementarity determining regions CDR1, CDR2, and CDR3, respectively.

Exemplarily, the amino acid sequence of nanobody PN7 is shown below:

(SEQ ID NO: 13)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSQKKITDYFEEFDYWGQGTLVTVSS

The underlined portions represent complementarity determining regions CDR1, CDR2, and CDR3, respectively.

Exemplarily, the amino acid sequence of nanobody PN8 is shown below:

(SEQ ID NO: 14)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSAVLQKKITDYFHPKKEEFDYWGQGTLVTVSS.

The underlined portions represent complementarity determining regions CDR1, CDR2, and CDR3, respectively.

The nucleotide sequences of the genes encoding nanobodies PN2, PN6, PN7, and PN8 are shown in SEQ ID NOs: 18-21, respectively.

### Example 2: Expression and Purification of Nanobodies and PCNA Antigen

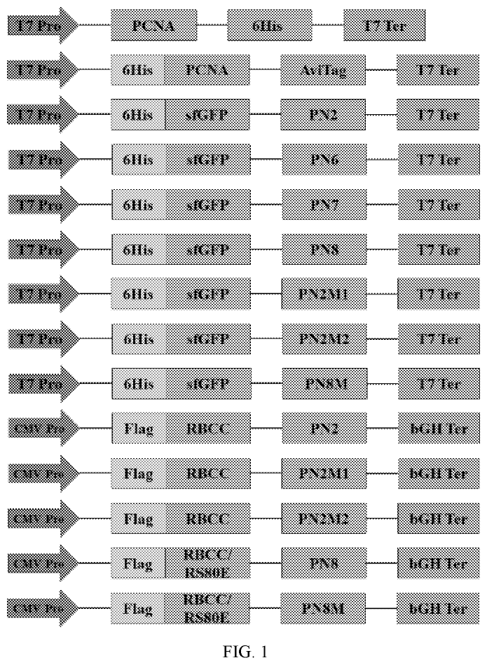

To facilitate the isolation and purification of nanobodies, in the present disclosure, the nanobodies prepared in Example 1 were displayed on the surface of Escherichia coli via superfolder green fluorescent protein (sfGFP). The nucleotide sequence of the gene encoding the sfGFP protein is shown in SEQ ID NO: 25. Specifically, using pET23a as the backbone vector, the genes encoding the aforementioned nanobodies were fused to the sfGFP gene via a first linker (the nucleotide sequence of which is shown in SEQ ID NO: 26). Expression vectors were constructed by homologous recombination. The element arrangement in these expression vectors is shown in FIG. 1. The recombinant plasmids were pET23a-sfGFP-PN2, pET23a-sfGFP-PN6, pET23a-sfGFP-PN7 and pET23a-sfGFP-PN8, respectively.

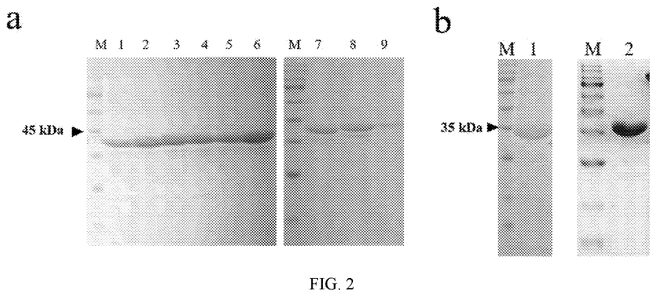

The constructed recombinant plasmids were separately transformed into E. coli competent cells BL21 (DE3), followed by static cultivation at 37° C. overnight to obtain a series of recombinant strains. Single colonies were then picked and inoculated into 100 mL of LB liquid medium (containing 50 μg/mL ampicillin) for shake cultivation at 37° C. When the OD₆₀₀ reached approximately 0.6, isopropyl β-D-1-thiogalactopyranoside (IPTG) was added to a final concentration of 0.5 mM, and induction was performed with shaking at 18° C. for 18 hours. After cultivation, the bacterial cells and the culture supernatant were separately collected by centrifugation at 12,000 rpm and 4° C. The bacterial cells were washed with TEN buffer (50 mM Tris-HCl, 5 mM EDTA, 50 mM NaCl, pH 8.0) at 4° C. for more than 4 hours, and the proteins secreted and expressed by the strains were directly obtained from the TEN buffer. SDS-PAGE was performed to detect and analyze the secretory expression of the nanobodies, and the results are shown in FIG. 2a.

As can be seen from FIG. 2a, nanobodies PN1-PN9 were all successfully expressed.

Representatively, the purified nanobodies PN2, PN6, PN7 and PN8 obtained by outer membrane washing were stored for subsequent experiments.

Further, the PCNA antigen sequence (adopting amino acids 1-261 of NCBI Accession No.: P12004) was cloned into the pET23a vector by homologous recombination to obtain the recombinant plasmid pET23a-PCNA (vector structure shown in FIG. 1). The constructed recombinant plasmid was transformed into E. coli competent cells BL21 (DE3). A single colony was picked and inoculated into 100 mL of LB liquid medium (containing 50 μg/mL ampicillin) for static cultivation at 37° C. overnight. When the OD600 reached approximately 0.6, IPTG was added to a final concentration of 0.5 mM, and induction was performed with shaking at 18° C. for 18 hours. The bacterial cells were collected by centrifugation at 6,000 rpm for 10 min, washed with phosphate-buffered saline (PBS), and then resuspended in PBS. Phenylmethylsulfonyl fluoride (PMSF) was added to a final concentration of 1 mM, followed by high-pressure disruption of the cells. The resulting lysate was centrifuged at 18,000 rpm for 30 min, and the supernatant was collected, filtered, and purified by Ni-NTA affinity chromatography. The purified protein was collected and detected by SDS-PAGE. It was then divided into small aliquots, snap-frozen in liquid nitrogen, and stored at −80° C. The identification and analysis results of SDS-PAGE are shown in FIG. 2b. The predicted molecular weight of PCNA was calculated to be 29.8 kDa via http://www.expasy.org/, and the results indicated that the PCNA antigen was successfully expressed.

### Example 3: ELISA Assay of Nanobodies

This example aims to verify whether the purified nanobodies PN2, PN6, PN7, and PN8 from Example 2 can directly interact with the purified PCNA antigen from Example 2. The detailed steps are as follows:
a) Diluting the PCNA antigen to 1 μg/mL with 1× ELISA coating buffer, adding the diluted antigen solution to a microplate at 100 μL/well, and incubating the microplate statically at 4° C. overnight;
b) Washing the microplate with PBST, and blocking the microplate with 1% BSA at room temperature for 2 h at 100 μL/well;
c) Preparing nanobodies PN2, PN6, PN7, and PN8 at various concentrations with 1% BSA, adding the nanobody solution to the microplate at 100 μL/well, and incubating the microplate at room temperature for 1 h;d) Incubating with HRP-conjugated Mouse anti-HA-Tag mAb (secondary antibody) at room temperature for 1 h;
e) Developing color with TMB substrate solution, and then terminating the reaction with stop solution;
f) Measuring the absorbance at 450 nm using a microplate reader.

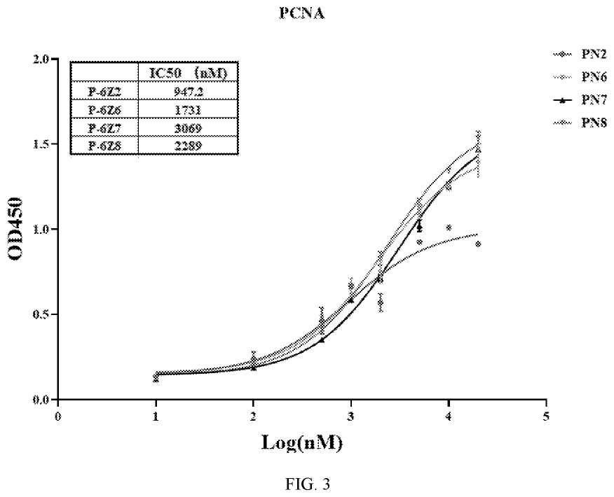

The test results are shown in FIG. 3. As can be seen from FIG. 3, nanobodies PN2, PN6, PN7, and PN8 exhibit favorable affinity for the PCNA antigen.

### Example 4: Bio-Layer Interferometry (BL1) Assay of Nanobodies

This example aims to further verify the binding between the purified nanobodies PN2, PN6, PN7, and PN8 from Example 2 and the purified PCNA antigen from Example 2, and to calculate the affinity between them.

Specifically, the PCNA antigen was immobilized onto a biosensor chip, and the nanobodies PN2, PN6, PN7, and PN8 at different concentrations were separately added to analyze their affinity for the antigen protein. The increase in optical thickness at the tip of the biosensor was recorded over 600 seconds. The affinity for PCNA was calculated according to the association and dissociation rates of the antibodies at different concentrations.

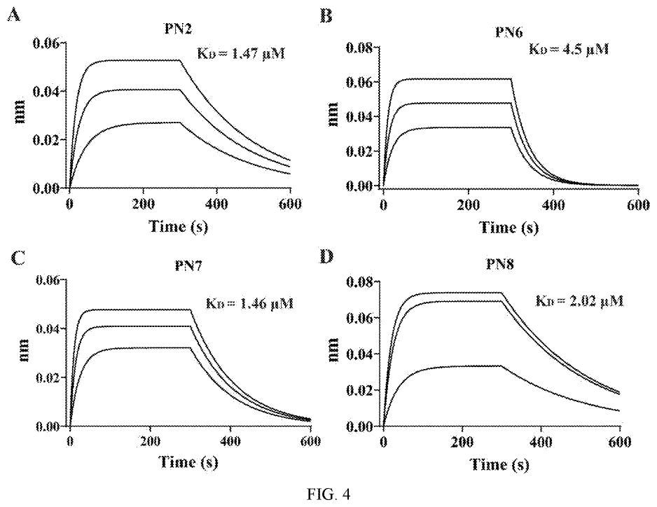

The results are shown in FIG. 4. As can be seen from FIG. 4, nanobodies PN2, PN6, PN7, and PN8 exhibit favorable affinity for the PCNA antigen.

### Example 5: Affinity Improvement Assay of Nanobodies

To further improve the affinity of the nanobodies, rational design of the nanobodies was performed using artificial intelligence in the present disclosure. Mutations were introduced into nanobody PN2 to obtain the following mutants (PN2M): PN2S101F (denoted as 2S101F), PN2S101G (denoted as 2S101G), PN2K104V (denoted as 2K104V), PN2K104R (denoted as 2K104R), PN2L102W (denoted as 2L102W), PN2S101M (denoted as 2S101M), PN2S101N (denoted as 2S101N), and PN2K104I (denoted as 2K104I). Mutations were introduced into nanobody PN8 to obtain the following mutants (PN8M): PN8H113K (denoted as 8H113K) and PN8T109L (denoted as 8T109L).

Furthermore, the binding energy and total score of PN2-WT and its mutant PN2M with the PCNA antigen were calculated using Rosetta. Similarly, the binding energy and total score of PN8-WT and its mutant PN8M with the PCNA antigen were calculated using Rosetta.

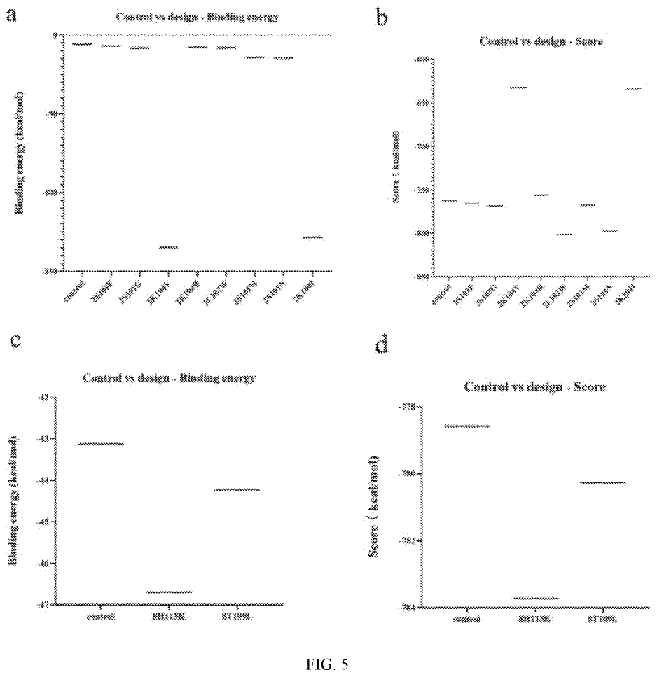

The results are shown in FIG. 5. As can be seen from FIG. 5, 2S101F, 2S101G, 2K104R, 2L102W, and 2S101M, etc., exhibit higher scores (FIG. 5b) and lower energy values (FIG. 5a), and 8H113K exhibits the highest score (FIG. 5d) and the lowest energy value (FIG. 5c).

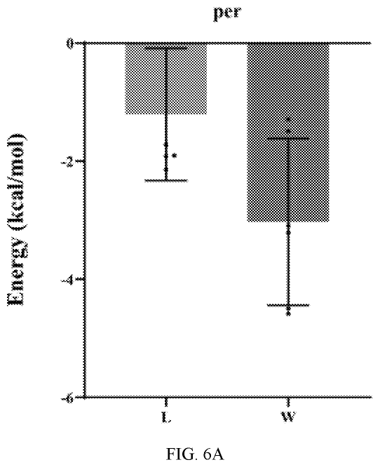

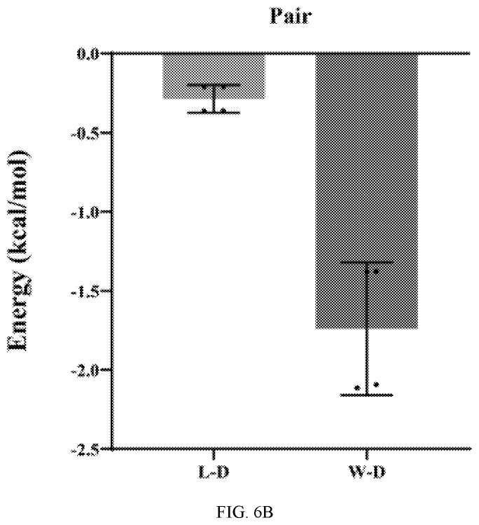

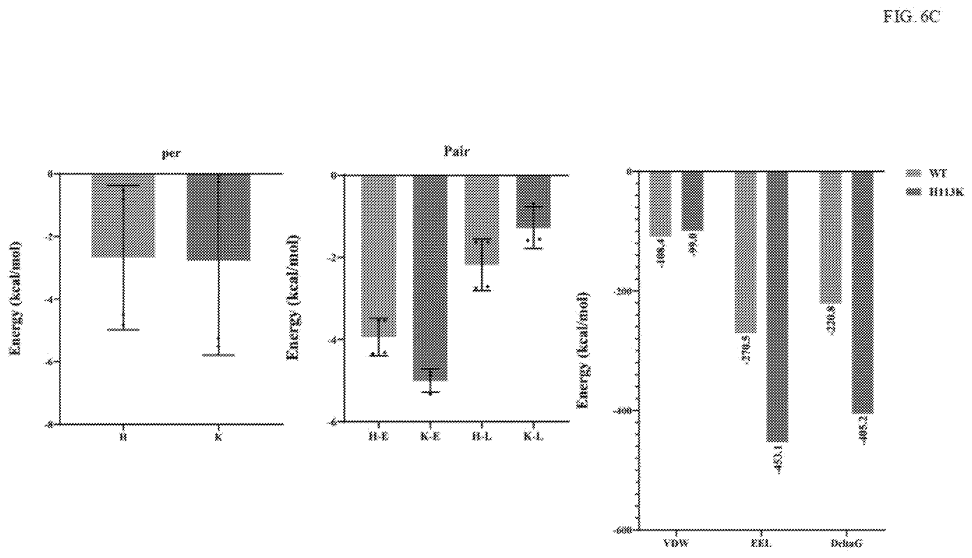

The individual energy change at the mutation site of PN2L102W was further calculated using Amber. It was found that mutation of L to W at position 102 in PN2L102W resulted in a lower energy value and better stability, indicating expected higher affinity (FIG. 6A). For complex pairing energy calculation, the overall energy of the paired amino acids with the mutated W was also lower than that of the original L (FIG. 6B).

The individual energy change at the mutation site of PN8H113K was further calculated using Amber. It was found that mutation of H to K at position 113 in PN8H113K resulted in a lower energy value and better stability. For complex pairing energy calculation, the overall energy of the paired amino acids with the mutated K was also lower than that of the original H (FIG. 6C).

Using the method described in Example 2, expression vectors for nanobody mutants PN2M and PN8M were constructed (see FIG. 1). The nanobody mutant PN2M includes 2S101F, 2S101G, 2K104V, 2K104R, 2L102W, 2S101M, 2S101N, and 2K104I. The nucleotide sequence of 2S101F is obtained by substituting nucleotides “AGT” at positions 301-303 of SEQ ID NO: 18 with “TTC”; the nucleotide sequence of 2S101G is obtained by substituting nucleotides “AGT” at positions 301-303 of SEQ ID NO: 18 with “GGC”; the nucleotide sequence of 2K104V is obtained by substituting nucleotides “AAA” at positions 310-312 of SEQ ID NO: 18 with “GTG”; the nucleotide sequence of 2K104R is obtained by substituting nucleotides “AAA” at positions 310-312 of SEQ ID NO: 18 with “AGA”; the nucleotide sequence of 2L102W is obtained by substituting nucleotides “CTG” at positions 304-306 of SEQ ID NO: 18 with “TGG”; the nucleotide sequence of 2S101M is obtained by substituting nucleotides “AGT” at positions 301-303 of SEQ ID NO: 18 with “ATG”; the nucleotide sequence of 2S101N is obtained by substituting nucleotides “AGT” at positions 301-303 of SEQ ID NO: 18 with “AAC”; the nucleotide sequence of 2K104I is obtained by substituting nucleotides “AAA” at positions 310-312 of SEQ ID NO: 18 with “ATT”. The nucleotide sequence of 8H113K is obtained by substituting nucleotides “CAC” at positions 337-339 of SEQ ID NO: 21 with “AAA”; the nucleotide sequence of 8T109L is obtained by substituting nucleotides “ACC” at positions 325-327 of SEQ ID NO: 21 with “CTG”.

Induction, expression and purification were then performed to obtain nanobody mutants 2S101F, 2S101G, 2K104V, 2K104R, 2L102W, 2S101M, 2S101N, 2K104I, 8H113K, and 8T109L.

Using the method described in Example 3, ELISA assays were performed to determine the affinity of the above-mentioned PN2 and its mutant PN2M, and PN8 and its mutant PN8M toward the PCNA antigen.

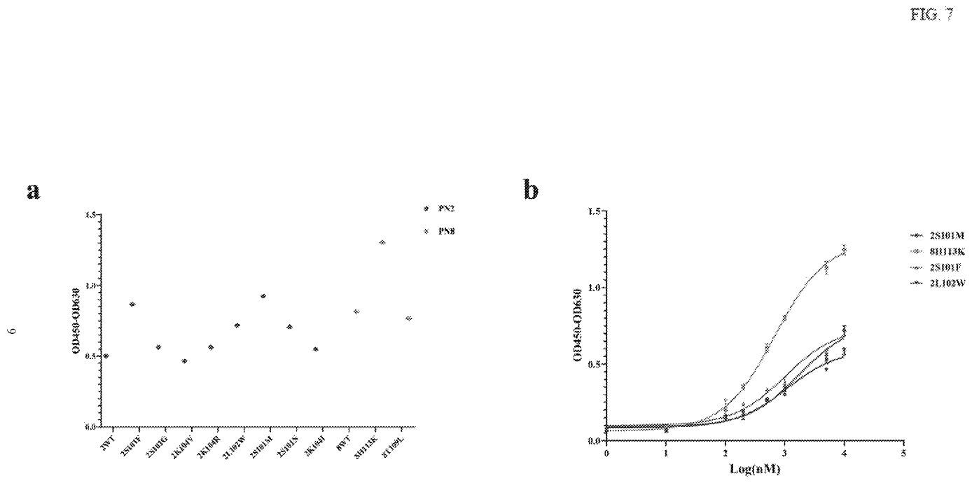

The results are shown in FIG. 7. As can be seen from FIG. 7, compared to nanobodies PN2 and PN8, PN2S101F, PN2L102W, PN2S101M, and PN8H113K exhibit improved affinity for the PCNA antigen.

Using the method described in Example 4, BL1 assays were performed to determine the affinity of nanobodies PN2, PN8 and nanobody mutants 2S101F, 2L102W, 2S101M, and 8H113K toward the PCNA antigen.

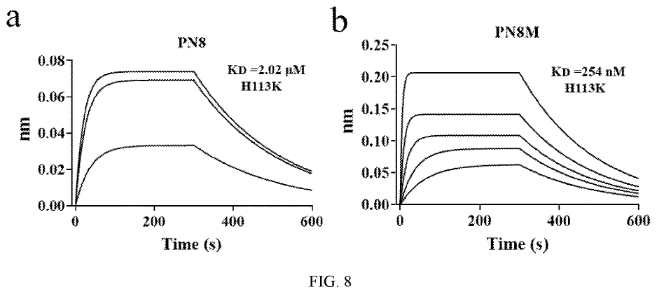

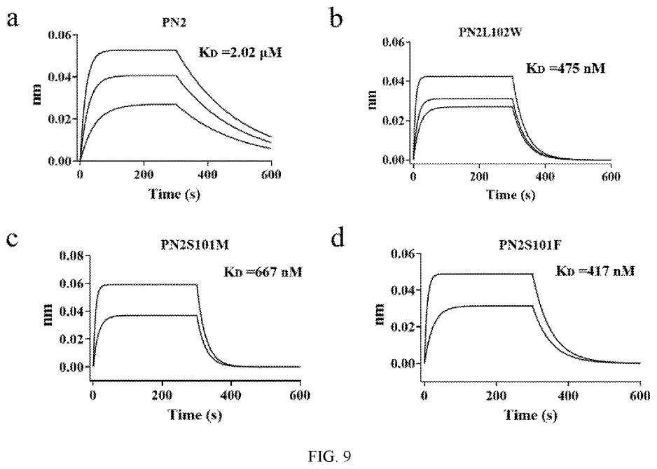

The results are shown in FIGS. 8 and 9. As can be seen from FIGS. 8 and 9, PN2S101F, PN2L102W, PN2S101M, and PN8H113K exhibit improved affinity for the PCNA antigen compared with nanobodies PN2 and PN8.

Finally, nanobody mutants 2L102W, 2S101M, and 8H113K with significantly improved affinity were obtained, and were designated as nanobody mutants PN2M1, PN2M2, and PN8M, respectively.

The amino acid sequence of nanobody mutant PN2M1 is shown below:

(SEQ ID NO: 15)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSWQKGYLEEFDYWGQGTLVTVSS.

The underlined portions represent complementarity determining regions CDR1, CDR2, and CDR3, respectively.

The amino acid sequence of nanobody mutant PN2M2 is shown below:

(SEQ ID NO: 16)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGMLQKGYLEEFDYWGQGTLVTVSS

The underlined portions represent complementarity determining regions CDR1, CDR2, and CDR3, respectively.

The amino acid sequence of nanobody mutant PN8M is shown below:

(SEQ ID NO: 17)
MEVQLEESGGGLVQPGGSLRLSCAASGFNIKDTYIGWVRQAPGKG
EEWVASIYPTSGYTRYADSVKGRFTISADTSKNTAYLQMNSLRAE
DTAVYYCAAGSAVLQKKITDYFKPKKEEFDYWGQGTLVTVSS

The underlined portions represent complementarity determining regions CDR1, CDR2, and CDR3, respectively.

The nucleotide sequences of the genes encoding nanobody mutants PN2M1, PN2M2, and PN8M are shown in SEQ ID NOs: 22-24, respectively.

### Example 6: Targeted Degradation of the PCNA Protein by Fusion Proteins

To achieve targeted degradation of the PCNA protein, in the present disclosure, the nanobodies PN2, PN6, PN7, PN8 and nanobody mutants PN2M1, PN2M2 and PN8M were respectively fused with the RBCC domain or its mutant RS79E to obtain fusion proteins. The fusion proteins were expressed in HEK293T cells, and the targeted degradation efficiency of PCNA protein by these fusion proteins was detected. Specifically, using pcDNA3.1 as the backbone vector, the genes encoding the aforementioned nanobodies PN2, PN6, PN7, PN8 and their mutants PN2M1, PN2M2 and PN8M were fused to the gene encoding the RBCC domain (the amino acid sequence of which is shown in SEQ ID NO: 27, and the nucleotide sequence of which is shown in SEQ ID NO: 31) via a second linker (the amino acid sequence of which is shown in SEQ ID NO: 28, and the nucleotide sequence of which is shown in SEQ ID NO: 32). Expression vectors were constructed by homologous recombination. The element arrangement in these expression vectors is shown in FIG. 1. The recombinant plasmids were pcDNA3.1-RBCC-PN2, pcDNA3.1-RBCC-PN6, pcDNA3.1-RBCC-PN7, pcDNA3.1-RBCC-PN8, pcDNA3.1-RBCC-PN2M1, pcDNA3.1-RBCC-PN2M2, pcDNA3.1-RBCC-PN8M, pcDNA3.1-RS79E-PN8 and pcDNA3.1-RS79E-PN8M, respectively.

The amino acid sequences of the fusion proteins RBCC-PN2, RBCC-PN6, RBCC-PN7, RBCC-PN8, RBCC-PN2M1, RBCC-PN2M2, RBCC-PN8M, RS79E-PN8 and RS79E-PN8M are shown in SEQ ID NOs: 33-41, respectively.

Exemplarily, using PEI reagent, the recombinant plasmids pcDNA3.1-RBCC-PN8, pcDNA3.1-RBCC-PN8M, pcDNA3.1-RS79E-PN8, pcDNA3.1-RS79E-PN8M, pcDNA3.1-RBCC-PN2M1, pcDNA3.1-RBCC-PN2M2 and a GFP plasmid were electroporated into well-growing HEK293T cells. After electroporation, the transfection efficiency and protein expression were monitored using the GFP signal.

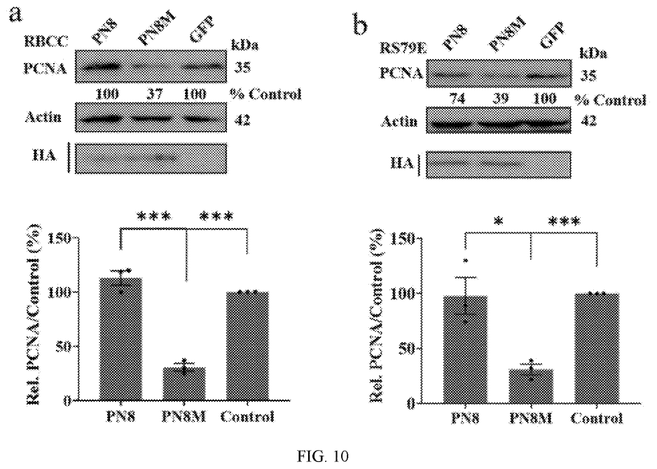

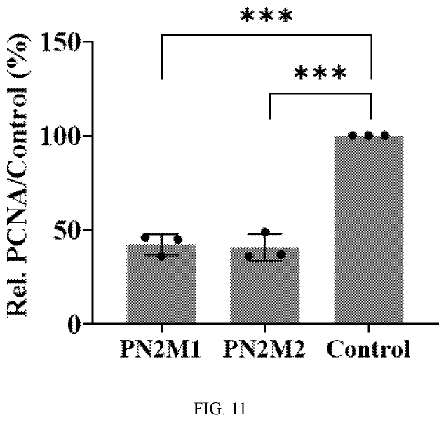

Cells were harvested 48 h post-transfection, and then subjected to Western blot (WB) analysis. Based on the WB results, the targeted protein degradation efficiency at 48 h post-transfection was calculated. The results are shown in FIGS. 10 and 11, respectively.

As can be seen from FIG. 10, the fusion proteins containing nanobody PN8 and nanobody mutant PN8M exhibit favorable degradation effects on the PCNA antigen, and the fusion protein containing nanobody mutant PN8M shows a PCNA antigen degradation efficiency of about 60%. Furthermore, when the RBCC domain mutant RS79E was fused with the nanobody mutant PN8M, a significantly enhanced degradation efficiency toward the PCNA antigen is observed.

As can be seen from FIG. 11, the fusion proteins containing nanobody mutants PN2M1 and PN2M2 exhibit favorable degradation effects on the PCNA antigen.

Finally, the degradation pathway by which the fusion proteins targetedly degrade the PCNA protein was investigated.

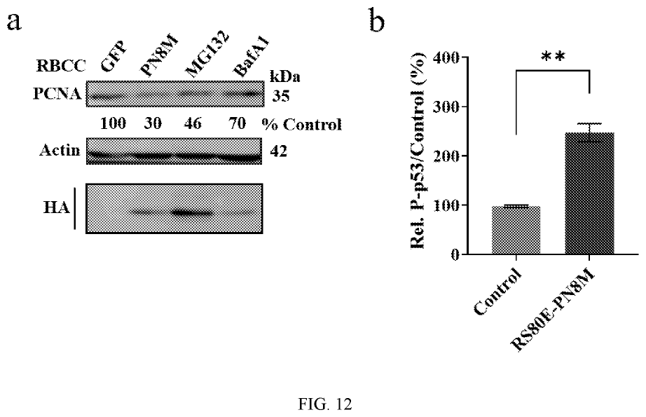

Specifically, MG132 proteasome inhibitor at a final concentration of 15 μM or BafA1 autophagy inhibitor at a final concentration of 200 nM was used, in which MG132 can inhibit the ubiquitin-proteasome pathway and BafA1 can inhibit the lysosomal pathway. Three samples of recombinant plasmid pcDNA3.1-RBCC-PN8M and a GFP plasmid were electroporated into well-growing HEK293T cells. At 24 h post-transfection, MG132 and BafA1 were added to two samples of the cells containing pcDNA3.1-RBCC-PN8M, respectively. After further incubation, the cells were subjected to Western blot analysis. The results are shown in FIG. 12.

As can be seen from FIG. 12, the targeted degradation of PCNA by the fusion proteins in the present disclosure occurs through both the ubiquitin-proteasome pathway and the lysosomal pathway, and primarily through the lysosomal pathway. Furthermore, compared with the control group, the level of phosphorylated p53 is significantly increased after targeted degradation of PCNA by RS79E-PN8M in the experimental group (FIG. 12b). The results demonstrate that the targeted degradation of the PCNA protein by the fusion proteins in the present disclosure is achieved via the ubiquitin-proteasome pathway or the autophagy-lysosomal pathway.

In summary, the present disclosure utilizes artificial intelligence technology to design nanobodies that specifically target PCNA. The nanobodies exhibit favorable specificity and high affinity, and are capable of efficiently binding to the PCNA antigen. Further, rational design of the nanobodies via artificial intelligence technology yields nanobody mutants with improved affinity. Moreover, fusion proteins are prepared by fusing the aforementioned nanobodies or nanobody mutants to the RBCC domain or its mutant. The fusion proteins can specifically bind to the target protein via their nanobody domains, activate the protein degradation pathway, and achieve targeted degradation of PCNA protein.

The foregoing examples merely represent certain embodiments of the present disclosure. The descriptions thereof are relatively specific and detailed, but should not be construed as limiting the scope of the present disclosure. It should be noted that, for those of ordinary skill in the art, any modifications, equivalent replacements and improvements made within the concepts and principles of the present disclosure shall fall within the protection scope of the present disclosure. Therefore, the scope of protection of the present disclosure shall be subject to the appended claims.

What is claimed is:

1. An anti-PCNA protein nanobody, comprising complementarity determining region 1 (CDR1), complementarity determining region 2 (CDR2) and complementarity determining region 3 (CDR3);
wherein the amino acid sequence of CDR1 is shown in SEQ ID NO: 1, the amino acid sequence of CDR2 is shown in SEQ ID NO: 2, and the amino acid sequence of CDR3 is shown in any one of SEQ ID NOs: 3-6.

2. The anti-PCNA protein nanobody according to claim 1, further comprising framework region 1 (FR1), framework region 2 (FR2), framework region 3 (FR3), and framework region 4 (FR4);
wherein the amino acid sequence of FR1 is shown in SEQ ID NO: 7, the amino acid sequence of FR2 is shown in SEQ ID NO: 8, the amino acid sequence of FR3 is shown in SEQ ID NO: 9, and the amino acid sequence of FR4 is shown in SEQ ID NO: 10.

3. The anti-PCNA protein nanobody according to claim 2, wherein the nanobody is any one selected from A1) and A4):
A1) an amino acid sequence as shown in any one of SEQ ID NOs: 11-14;
A4) an amino acid sequence obtained by linking a tag or a signal peptide to the N-terminus and/or C-terminus of the amino acid sequence defined in A1).

4. A mutant of an anti-PCNA protein nanobody, wherein the mutant nanobody is any one selected from B1) and B4):
B1) an amino acid sequence as shown in any one of SEQ ID NOs: 15-17;
B4) an amino acid sequence obtained by linking a tag or a signal peptide to the N-terminus and/or C-terminus of the amino acid sequence defined in B1);
wherein, in B1), the amino acid sequence as shown in SEQ ID NO: 15 is obtained by mutating the amino acid sequence as shown in SEQ ID NO: 11 at position 102 from L to W (L102W), the amino acid sequence as shown in SEQ ID NO: 16 is obtained by mutating the amino acid sequence as shown in SEQ ID NO: 11 at position 101 from S to M (S101M), the amino acid sequence as shown in SEQ ID NO: 17 is obtained by mutating the amino acid sequence as shown in SEQ ID NO: 14 at least at position 113 from H to K (H113K).

5. A fusion protein, wherein the fusion protein is prepared by fusing the mutant nanobody according to claim 4 to an RBCC domain or its mutant via a second linker;
wherein the amino acid sequence of the second linker is shown in SEQ ID NO: 29, the amino acid sequence of the RBCC domain is shown in SEQ ID NO: 27, the amino acid sequence of the RBCC domain mutant is shown in SEQ ID NO: 28; and the amino acid sequence of the fusion protein is shown in any one of SEQ ID NOs: 37, 38, 39, and 41.

6. A biological material selected from any one of D1) to D3):
D1) a nucleic acid molecule encoding the nanobody according to claim 1, a mutant nanobody thereof, or a fusion protein comprising the mutant nanobody;
wherein the mutant nanobody is any one selected from B1) and B4):
B1) an amino acid sequence as shown in any one of SEQ ID NOs: 15-17;
B4) an amino acid sequence obtained by linking a tag or a signal peptide to the N-terminus and/or C-terminus of the amino acid sequence defined in B1);
in B1), the amino acid sequence as shown in SEQ ID NO: 15 is obtained by mutating the amino acid sequence as shown in SEQ ID NO: 11 at position 102 from L to W (L102W), the amino acid sequence as shown in SEQ ID NO: 16 is obtained by mutating the amino acid sequence as shown in SEQ ID NO: 11 at position 101 from S to M (S101M), the amino acid sequence as shown in SEQ ID NO: 17 is obtained by mutating the amino acid sequence as shown in SEQ ID NO: 14 at least at position 113 from H to K (H113K);
wherein the fusion protein is prepared by fusing the mutant nanobody to an RBCC domain or its mutant via a second linker, the amino acid sequence of the second linker is shown in SEQ ID NO: 29, the amino acid sequence of the RBCC domain is shown in SEQ ID NO: 27, the amino acid sequence of the RBCC domain mutant is shown in SEQ ID NO: 28; and the amino acid sequence of the fusion protein is shown in any one of SEQ ID NOs: 37, 38, 39, and 41;
D2) a recombinant vector comprising the nucleic acid molecule of D1);
D3) a recombinant cell comprising the nucleic acid molecule of D1) or the recombinant vector of D2).

7. The biological material according to claim 6, wherein the nanobody further comprises framework region 1 (FR1), framework region 2 (FR2), framework region 3 (FR3), and framework region 4 (FR4);
wherein the amino acid sequence of FR1 is shown in SEQ ID NO: 7, the amino acid sequence of FR2 is shown in SEQ ID NO: 8, the amino acid sequence of FR3 is shown in SEQ ID NO: 9, and the amino acid sequence of FR4 is shown in SEQ ID NO: 10.

8. The biological material according to claim 7, wherein the nanobody is any one selected from A1) and A4):
A1) an amino acid sequence as shown in any one of SEQ ID NOs: 11-14;
A4) an amino acid sequence obtained by linking a tag or a signal peptide to the N-terminus and/or C-terminus of the amino acid sequence defined in A1).

9. A pharmaceutical composition, comprising any one selected from F1) to F5), and a pharmaceutically acceptable carrier;
F1) the nanobody according to claim 1;
F2) a mutant of the nanobody of F1), wherein the mutant nanobody is any one selected from B1) and B4):
B1) an amino acid sequence as shown in any one of SEQ ID NOs: 15-17;
B4) an amino acid sequence obtained by linking a tag or a signal peptide to the N-terminus and/or C-terminus of the amino acid sequence defined in B1);
in B1), the amino acid sequence as shown in SEQ ID NO: 15 is obtained by mutating the amino acid sequence as shown in SEQ ID NO: 11 at position 102 from L to W (L102W), the amino acid sequence as shown in SEQ ID NO: 16 is obtained by mutating the amino acid sequence as shown in SEQ ID NO: 11 at position 101 from S to M (S101M), the amino acid sequence as shown in SEQ ID NO: 17 is obtained by mutating the amino acid sequence as shown in SEQ ID NO: 14 at least at position 113 from H to K (H113K);
F3) a fusion protein prepared by fusing the mutant nanobody of F2) to an RBCC domain or its mutant via a second linker; wherein the amino acid sequence of the second linker is shown in SEQ ID NO: 29, the amino acid sequence of the RBCC domain is shown in SEQ ID NO: 27, the amino acid sequence of the RBCC domain mutant is shown in SEQ ID NO: 28; and the amino acid sequence of the fusion protein is shown in any one of SEQ ID NOs: 37, 38, 39, and 41;
F4) a biological material selected from any one of D1) to D3):
D1) a nucleic acid molecule encoding the nanobody of F1), the mutant nanobody of F2), or the fusion protein of F3);
D2) a recombinant vector comprising the nucleic acid molecule of D1);
D3) a recombinant cell comprising the nucleic acid molecule of D1) or the recombinant vector of D2).

10. The pharmaceutical composition according to claim 9, wherein the nanobody further comprises framework region 1 (FR1), framework region 2 (FR2), framework region 3 (FR3), and framework region 4 (FR4);
wherein the amino acid sequence of FR1 is shown in SEQ ID NO: 7, the amino acid sequence of FR2 is shown in SEQ ID NO: 8, the amino acid sequence of FR3 is shown in SEQ ID NO: 9, and the amino acid sequence of FR4 is shown in SEQ ID NO: 10.

11. The pharmaceutical composition according to claim 10, wherein the nanobody is any one selected from A1) and A4):
A1) an amino acid sequence as shown in any one of SEQ ID NOs: 11-14;
A4) an amino acid sequence obtained by linking a tag or a signal peptide to the N-terminus and/or C-terminus of the amino acid sequence defined in A1).

12. A method for preparing the nanobody according to claim 1, a mutant nanobody thereof, or a fusion protein comprising the mutant nanobody, comprising:
culturing a recombinant cell to obtain a culture after induced expression; and
isolating the nanobody, the nanobody mutant, or the fusion protein from the culture;
wherein the recombinant cell comprises a nucleic acid molecule encoding the nanobody, the mutant nanobody, or the fusion protein, or the recombinant cell comprises a recombinant vector that comprises said nucleic acid molecule;
wherein the mutant nanobody is any one selected from B1) and B4):
B1) an amino acid sequence as shown in any one of SEQ ID NOs: 15-17;
B4) an amino acid sequence obtained by linking a tag or a signal peptide to the N-terminus and/or C-terminus of the amino acid sequence defined in B1);
in B1), the amino acid sequence as shown in SEQ ID NO: 15 is obtained by mutating the amino acid sequence as shown in SEQ ID NO: 11 at position 102 from L to W (L102W), the amino acid sequence as shown in SEQ ID NO: 16 is obtained by mutating the amino acid sequence as shown in SEQ ID NO: 11 at position 101 from S to M (S101M), the amino acid sequence as shown in SEQ ID NO: 17 is obtained by mutating the amino acid sequence as shown in SEQ ID NO: 14 at least at position 113 from H to K (H113K);
wherein the fusion protein is prepared by fusing the mutant nanobody to an RBCC domain or its mutant via a second linker, the amino acid sequence of the second linker is shown in SEQ ID NO: 29, the amino acid sequence of the RBCC domain is shown in SEQ ID NO: 27, the amino acid sequence of the RBCC domain mutant is shown in SEQ ID NO: 28; and the amino acid sequence of the fusion protein is shown in any one of SEQ ID NOs: 37, 38, 39, and 41.

13. The method according to claim 12, wherein the nanobody further comprises framework region 1 (FR1), framework region 2 (FR2), framework region 3 (FR3), and framework region 4 (FR4);
wherein the amino acid sequence of FR1 is shown in SEQ ID NO: 7, the amino acid sequence of FR2 is shown in SEQ ID NO: 8, the amino acid sequence of FR3 is shown in SEQ ID NO: 9, and the amino acid sequence of FR4 is shown in SEQ ID NO: 10.

14. The method according to claim 13, wherein the nanobody is any one selected from A1) and A4):
A1) an amino acid sequence as shown in any one of SEQ ID NOs: 11-14;
A4) an amino acid sequence obtained by linking a tag or a signal peptide to the N-terminus and/or C-terminus of the amino acid sequence defined in A1).


SEQUENCE LISTING (41 sequences)

SEQ ID NO: 1 - protein, 7 aa, synthetic construct
GFNIKDT

SEQ ID NO: 2 - protein, 6 aa, synthetic construct
YPTSGY

SEQ ID NO: 3 - protein, 13 aa, synthetic construct
GSLQKGYLEEFDY

SEQ ID NO: 4 - protein, 16 aa, synthetic construct
GSAVLQKGYFPEEFDY

SEQ ID NO: 5 - protein, 15 aa, synthetic construct
GSQKKITDYFEEFDY

SEQ ID NO: 6 - protein, 22 aa, synthetic construct
GSAVLQKKITDYFHPKKEEFDY

SEQ ID NO: 7 - protein, 26 aa, synthetic construct
MEVQLEESGGGLVQPGGSLRLSCAAS

SEQ ID NO: 8 - protein, 19 aa, synthetic construct
YIGWVRQAPGKGEEWVASI

SEQ ID NO: 9 - protein, 41 aa, synthetic construct
TRYADSVKGRFTISADTSKNTAYLQMNSLRAEDTAVYYCAA

SEQ ID NO: 10 - protein, 11 aa, synthetic construct
WGQGTLVTVSS

SEQ ID NO: 11 - protein, 123 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSLQKGYLEEFDYWGQGTLVTVSS

SEQ ID NO: 12 - protein, 126 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKGYFPEEFDYWGQGTLVTVSS

SEQ ID NO: 13 - protein, 125 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSQKKITDYFEEFDYWGQGTLVTVSS

SEQ ID NO: 14 - protein, 132 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFHPKKEEFDYWGQGTLVTVSS

SEQ ID NO: 15 - protein, 123 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSWQKGYLEEFDYWGQGTLVTVSS

SEQ ID NO: 16 - protein, 123 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGMLQKGYLEEFDYWGQGTLVTVSS

SEQ ID NO: 17 - protein, 132 aa, synthetic construct
MEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFKPKKEEFDYWGQGTLVTVSS

SEQ ID NO: 18 - DNA, 369 bp, synthetic construct
  source 1..369: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtctgcagaaaggctatctggaggaatttgactattggggccagggcaccttagttaccgttagctcg

SEQ ID NO: 19 - DNA, 378 bp, synthetic construct
  source 1..378: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtgcggtgctgcagaaaggctattttccggaggaatttgactattggggccagggcaccttagttaccgttagctcg

SEQ ID NO: 20 - DNA, 375 bp, synthetic construct
  source 1..375: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtcaaaagaaaatcaccgattacttcgaggaatttgactattggggccagggcaccttagttaccgttagctcg

SEQ ID NO: 21 - DNA, 396 bp, synthetic construct
  source 1..396: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtgcggtgctgcaaaagaagatcaccgattacttccacccgaagaaagaggaatttgactattggggccagggcaccttagttaccgttagctcg

SEQ ID NO: 22 - DNA, 369 bp, synthetic construct
  source 1..369: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagttggcagaaaggctatctggaggaatttgactattggggccagggcaccttagttaccgttagctcg

SEQ ID NO: 23 - DNA, 369 bp, synthetic construct
  source 1..369: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcatgctgcagaaaggctatctggaggaatttgactattggggccagggcaccttagttaccgttagctcg

SEQ ID NO: 24 - DNA, 396 bp, synthetic construct
  source 1..396: mol_type = other DNA
atggaagttcagctggaagaaagcggcggggggctggtgcagccgggtggtagcttacggctgagctgtgcggccagcggctttaacattaaagacacctacattggttgggttcggcaggcaccgggtaaaggggaagaatgggttgcaagcatttatccgacgagtggatatacacgttatgcagattcagttaaaggtcgctttacaatttcagcagacaccagcaaaaataccgcatatctgcagatgaatagtctgcgtgcagaagacaccgcagtttattattgtgcagcaggcagtgcggtgctgcaaaagaagatcaccgattacttcaaaccgaagaaagaggaatttgactattggggccagggcaccttagttaccgttagctcg

SEQ ID NO: 25 - DNA, 716 bp, synthetic construct
  source 1..716: mol_type = other DNA
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccgaatttgaaattcgtcataatgtggaagatggcagcgtgcagctggcggatcattatcagcagaataccccgattggcgatggcccagtgctgctgccggatgaccactatctgagcaccgaaagcgtgctgagcaaagatccgaatgaagatcgtgatcatatggtcctgctggaatttgtgaccgcggcaggcattgatctgggcatggatgaactgtataa

SEQ ID NO: 26 - DNA, 24 bp, synthetic construct
  source 1..24: mol_type = other DNA
ttggaggttttgttccagggtcca

SEQ ID NO: 27 - protein, 234 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 28 - protein, 234 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 29 - protein, 16 aa, synthetic construct
GGGGSYPYDVPDYAGS

SEQ ID NO: 30 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcagccaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 31 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcgaacaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 32 - DNA, 48 bp, synthetic construct
  source 1..48: mol_type = other DNA
ggcggaggtggcagctatccgtatgatgttccggattatgcaggaagc

SEQ ID NO: 33 - protein, 373 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSLQKGYLEEFDYWGQGTLVTVSS

SEQ ID NO: 34 - protein, 376 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKGYFPEEFDYWGQGTLVTVSS

SEQ ID NO: 35 - protein, 375 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSQKKITDYFEEFDYWGQGTLVTVSS

SEQ ID NO: 36 - protein, 382 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFHPKKEEFDYWGQGTLVTVSS

SEQ ID NO: 37 - protein, 373 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSWQKGYLEEFDYWGQGTLVTVSS

SEQ ID NO: 38 - protein, 373 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGMLQKGYLEEFDYWGQGTLVTVSS

SEQ ID NO: 39 - protein, 382 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFKPKKEEFDYWGQGTLVTVSS

SEQ ID NO: 40 - protein, 382 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFHPKKEEFDYWGQGTLVTVSS

SEQ ID NO: 41 - protein, 382 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMEVQLEESGGGLVQPGGSLRLSCAASGFNIKDTYIGWVRQAPGKGEEWVASIYPTSGYTRYADSVKGRFTISADTSKNTAYLQMNSLRAEDTAVYYCAAGSAVLQKKITDYFKPKKEEFDYWGQGTLVTVSS